(19) United States
(12) Patent Application Publication
(10) Pub. No.: US 2005/0112709 A1
(43) Pub. Date: May 26, 2005

(54) Histone h3methyltransferase polypeptide

(76) Inventors: Daniel Wolf, Central Scotland (GB); Tony Kouzarides, Cambridgeshire (GB)

Correspondence Address: NIXON & VANDERHYE, PC, 1100 N GLEBE ROAD, 8TH FLOOR, ARLINGTON, VA, 22201-4714, US

(21) Appl. No.: 10/497,316

(22) PCT Filed: Dec. 3, 2002
(86) PCT No.: PCT/GB02/05444

(30) Foreign Application Priority Data
Dec. 3, 2001 (GB) 0128964.4

(51) Int. Cl. G01N 33/53; G01N 33/537; G01N 33/543

(52) U.S. Cl. 435/7.92

(57) ABSTRACT

This invention relates to histone methylation, in particular to the characterisation of a human polypeptide which is found to be responsible for the methylation of lysine 9 of histone H3. Various methods and means related to the methylation of this residue and its effect on transcriptional activation are provided.

This invention relates to histone methylation, in particular to an enzyme responsible for the methylation of a specific residue on histone H3.

DNA in the eukaryotic nucleus is wrapped around a histone-core, which is a protein complex involving the four histones H4, H3, H2B and H2A. This DNA-histone structure (nucleosome) is not compatible with gene expression. Re-organisation of the nucleosome is required for transcription factors and RNA polymerase to have access to the DNA for transcription.

Covalent post-translational modifications of the amino-terminal tails of histones regulate the transcriptional ‘on’ or ‘off’ states of chromatin and influence chromosome condensation and segregation. Such modifications include acetylation, phosphorylation and methylation.

Suv39H1 is a methyltransferase which is specific for lysine 9 of histone H3 (Rea et al (2000) Nature 406 593-598). Methylation of lysine 9 by Suv39H1 leads to the recruitment of the HP1 repressor protein (Bannister et al (2001) Nature 410 120-124, Lachner et al (2001) 410 116-120) and the formation of transcriptionally silent heterochromatin.

Suv39H1 has homologues in human (Suv39H2), Drosophila (su(var)3-9) and S. pombe (clr4). Defects in Suv39H1 and its homologues have been correlated with defects In transcription and aberrant mitotic division and chromosomal mis-segregation.

Expression of the Suv39h1 protein has also been shown to inhibit cell proliferation of mammalian cells and its phosphorylation status is known to change as cells progress from G1 to S.

Yeast Clr4 (the S. pombe homologue of Suv39H1) has a histone methylase activity that has been shown to be essential for transcriptional repression (Bannister et al (2001) Nature 410 120-124). Lack of Clr4 methylase activity activates heterochromatically repressed genes at centromeres and may lead to chromosome mis-segregation.

The Suv39H1 methyltransferase activity is dependent on an evolutionarily conserved domain called a SET domain (SUV39 Enhancer of Zest and Trithorax). However, SET domains are also found in other proteins which do not have histone methyltransferase activity (Rea et al. (2000)). There are at least 73 SET domain-containing proteins in the human proteins database, many of which are not associated with histone methyltransferase activity.

The present inventors have identified that a previously uncharacterised human protein is a histone H3 methyltransferase. This protein, which contains a SET domain, is the subject of GenBank accession number NP_{—}036564, and is herein designated ESET.

The present inventors have further discovered that this methyltransferase has an activity which is similar to that of Suv39H1, in that it specifically methylates lysine 9 of histone H3.

Various aspects of the present invention provide for the use of an ESET polypeptide and a histone H3 polypeptide in screening methods and assays for agents which modulate methylation of histone H3 by the ESET polypeptide, and which may therefore be useful in the modulating cellular proliferation, for example in treating conditions such as cancer.

In a general aspect, the present invention provides an assay method for an agent with ability to modulate, e.g. disrupt, interfere with, or increase interaction and/or binding of histone H3 with an ESET polypeptide, the method including:
(a) bringing into contact an ESET polypeptide and a histone H3 polypeptide; and
(b) determining binding and/or interaction between the histone H3 polypeptide and the ESET polypeptide.

An assay may be carried out in the presence of a test compound under conditions in which, in the absence of the test compound, the ESET polypeptide will interact or bind with histone H3.

A method for identifying and/or obtaining a compound which modulates the interaction and/or binding of histone H3 with an ESET polypeptide may include:
(a) contacting an ESET polypeptide and a histone H3 polypeptide in the presence of a test compound; and
(b) determining binding and/or interaction between the histone H3 polypeptide and the ESET polypeptide.

The polypeptides may be contacted in the presence and absence of test compound and the binding and/or interaction determined. An increase or decrease in binding and/or interaction in the presence, relative to the absence of test compound is indicative that the test compound is a modulator of the interaction. For example, an increase in binding and/or interaction in the presence relative to the absence of test compound may be indicative that the test compound is an enhancer or potentiator of the interaction and a decease in binding and/or interaction in the presence relative to the absence of test compound may be indicative that the test compound is an inhibitor of the binding and/or interaction.

A test compound which stabilises the binding and/or interaction between the ESET polypeptide and the histone H3 polypeptide, and which may up-regulate ESET activity, may be obtained using conditions that are too harsh for the ESET polypeptide to bind to the histone H3 polypeptide in the absence of the compound.

A test compound which disrupts the interaction and which may down-regulate ESET activity, may be obtained under conditions in which the ESET polypeptide normally binds to the histone H3 polypeptide.

Those of skill in the art may vary the precise format of the assay of the invention using routine skill and knowledge For example, the interaction between the polypeptides may be studied in vitro by labelling one with a detectable label and bringing it into contact with the other which has been immobilised on a solid support. Suitable detectable labels include ³⁵S-methionine which may be incorporated into recombinantly produced peptides and polypeptides. Recombinantly produced peptides and polypeptides may also be expressed as a fusion protein containing an epitope which can be labelled with an antibody.

Fusion proteins may be generated that incorporate six histidine residues at either the N-terminus or C-terminus of the recombinant protein. Such a histidine tag may be used for purification of the protein by using commercially available columns which contain a metal ion, either nickel or cobalt (Clontech, Palo Alto, Calif., USA). These tags also serve for detecting the protein using commercially available monoclonal antibodies directed against the six histidine residues (Clontech, Palo Alto, Calif., USA).

The protein which is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

A method according to the invention may also take the form of an in vivo method. The in vivo method may be performed in a cell line such as a yeast strain in which the relevant polypeptides or peptides are expressed from one or more vectors introduced into the cell.

Various methods and uses of modulators which inhibit, potentiate, increase or stimulate methylation of histone H3 by an ESET polypeptide are provided as further aspects of the present invention.

The purpose of disruption, interference with or modulation of the methylation of histone H3 by an ESET polypeptide may be to modulate cellular functions such as transcription and proliferation which are mediated by virtue of such methylation, such as transcription, as discussed above and further below.

A method of screening for, identifying and/or obtaining a compound which modulates activity of an ESET polypeptide may include contacting one or more test compounds or agents with the ESET polypeptide in a suitable reaction medium, testing is the activity of the treated polypeptide and comparing that activity with the activity of the polypeptide in comparable reaction medium untreated with the test compound, compounds or agent. A difference in activity between the treated and untreated ESET polypeptides is indicative of a modulating effect of the relevant test compound or compounds.

Thus in another aspect, the invention provides an assay method for an agent with ability to modulate, e.g. disrupt, interfere with, increase or stimulate the histone H3 methylase activity of an ESET polypeptide, the method including:
(a) bringing into contact an ESET polypeptide and a test compound; and,
(b) determining the histone H3 methylase activity of the ESET polypeptide.

A method for identifying and/or obtaining a compound which modulates the histone H3 methylase activity of an ESET polypeptide may include:
(a) contacting an ESET polypeptide and a test compound; and,
(b) determining the histone H3 methylase activity of the ESET polypeptide.

The histone H3 methylase activity may be determined in the presence and absence of test compound. An increase or decrease in activity in the presence, relative to the absence of test compound is indicative that the test compound is a modulator of activity.

A method may be carried out under conditions in which, in the absence of a test compound with antagonist activity, the ESET polypeptide methylates the lysine 9 residue of histone H3. Alternatively, method may be carried out under conditions in is which, in the absence of a test compound with agonist activity, the ESET polypeptide does not methylate the lysine 9 residue of histone H3. The methylation of a histone H3 polypeptide by an ESET polypeptide may be determined as described herein.

The lysine 9 residue of histone H3 may be mono-, di- or tri-methylated by the ESET polypeptide.

In another aspect, the present invention provides an assay method for an agent with ability to modulate, e.g. disrupt, interfere with, increase or stimulate methylation of histone H3 by an ESET polypeptide, the method including:
(a) bringing into contact an ESET polypeptide and a histone H3 polypeptide in the presence of a test compound; and,
(b) determining methylation of the histone H3 polypeptide.

A method may be carried out under conditions in which, in the absence of the test compound, the ESET polypeptide will methylate histone H3.

Usually, methylation of the lysine 9 residue of histone H3 will be determined, for example, mono-, di- or tri-methylation of the lysine 9 residue.

A test compound which increases, potentiates, stimulates, disrupts, reduces, interferes with or wholly or partially abolishes methylation of the histone H3 polypeptide and which may thereby modulate the activity of the ESET polypeptide, may thus be identified and/or obtained.

Methylation may be determined according to known methods described herein.

Compounds and agents that increase or potentiate the methylation of histone H3 by an ESET polypeptide may be identified using conditions which, in the absence of a positively-acting agent, prevent methylation. Such compounds may be used to potentiate the function of an ESET polypeptide, and may have an effect for example, on transcription and/or DNA replication.

In methods of the invention, the histone H3 polypeptide may be contacted with the ESET polypeptide in the presence of a suitable substrate such as SAM (S-adenosyl-(methyl)-L-methionine). The substrate may be labelled, e.g. (S-adenosyl-(methyl-¹⁴C)-L-methionine) and the amount of label on the histone H3 after incubation with the ESET polypeptide determined.

The amount of test compound or compound which may be added to a method of the invention will normally be determined by trial and error depending upon the type of compound used. Typically, from about 0.001 nM to 1 mM or more concentrations of putative inhibitor compound may be used, for example from 0.01 nM to 100 μM, e.g. 0.1 to 50 μM, such as about 10 μM.

A test compound, compound or agent used in an assay method as described herein may be comprised in a sample, mixture or extract, for example, a biological sample.

A method may include purifying and/or isolating a test compound and/or compound of interest from a mixture or extract, i.e. reducing the content of at least one component is of the mixture or extract, e.g. a component with which the test compound is naturally associated. The method may include determining the ability of one or more fractions of a test mixture or extract to modulate the methylase activity of the ESET polypeptide.

The purification and/or isolation may employ any method known to those skilled in the art.

Those of skill in the art may vary the precise format of any of the methods of the invention using routine skill and knowledge. The skilled person is well aware of the need to employ appropriate control experiments.

Compounds which may be screened using the assay methods described herein may be natural or synthetic chemical compounds used in drug screening programmes. Extracts of plants, microbes or other organisms which contain several characterised or uncharacterised components may also be used.

Combinatorial library technology provides an efficient way of testing a potentially vast number of different compounds for ability to modulate an interaction. Such libraries and their use are known in the art, for all manner of natural products, small molecules and peptides, among others. The use of peptide libraries may be preferred in certain circumstances.

Methods of determining the methylation of histone H3 by an ESET polypeptide, for example mono-, di- or tri-methylation, and of obtaining and/or identifying a compound which modulates the methylation of histone H3 by an ESET polypeptide, include methods in which a suitable end-point is used to assess interaction.

Suitable end points include the determination of the methylation of the lysine 9 residue of histone H3 using methods as described herein.

Methylation may be determined by any convenient method known to a skilled person. For example, histone H3 polypeptide or variant or derivative thereof, may be immobilised e.g. on a bead or plate, and methylation of the lysine 9 residue detected using an antibody or other binding molecule which binds the N terminal region of histone H3 with a different affinity when the residue is methylated from when the residue is not methylated. In some embodiments, the antibody may be specific for the histone H3 polypeptide with a mono-, di- or tri-methylated lysine 9 residue. Such antibodies may be obtained by means of any standard technique as discussed elsewhere herein, e.g. using a methylated peptide (such as an N terminal fragment of histone H3).

Binding of a molecule which discriminates between the methylated and non-methylated form of a histone H3 polypeptide, or the mono-, di- and/or tri-methylated forms and other forms of histone H3, may be assessed using any technique available to those skilled in the art, which may involve determination of the presence of a suitable label.

Methylation may also be assayed in solution, e.g. as described in Rea et al (2000), Nature, 406: 593-599. Briefly, 10 μg of free histone substrate (mixture of H1, H2, H3, and H4; Boehringer Mannheim) is mixed with 300 nCi S-adenosyl-[methyl-¹⁴C]-L-methionine (25:Ci ml⁻¹) (Amersham) as methyl donor in methylase activity buffer (50 mM Tris.HCl pH8.5, 20 mM KCl, 10 mM MgCl₂, 10 mM β-mercaptoethanol, 250 mM sucrose), to give a final volume of 50 μl. 10:g of ESET polypeptide preparation is added and the reaction incubated at 37° C. for 60 mins. The reaction products are then resolved by SDS-PAGE and viewed following fluorography of the gel. Alternatively, following SDS-PAGE, the resolved proteins can be Western blotted to a nitrocellulose membrane, which is then dried and exposed to film.

Methylation may also be determined as described below in the examples.

Of course, the person skilled in the art will design any appropriate control experiments with which to compare results obtained in test assays.

Methods of determining the presence of, and optionally quantifying the amount of the ESET polypeptide in a test sample may have a diagnostic or prognostic purpose, e.g. in the diagnosis or prognosis or any medical condition discussed herein (e.g. a proliferative disorder such as cancer) or in the evaluation of a therapy to treat such a condition.

The characterisation of the histone H3 methylase activity of ESET polypeptides as described herein and its role in the regulation of cellular proliferation allows the use of materials and methods, such as are disclosed and discussed above, for establishing the presence or absence in a test sample, for example, obtained from an individual, of aberrant, i.e. increased, reduced or abolished ESET polypeptide histone H3 methylase activity. Such aberrant activity may determined as described herein for the purpose of diagnosing a predisposition of an individual to a condition associated with cellular proliferation or for diagnosing an individual as suffering from a condition associated with cellular proliferation, such as cancer. The presence of aberrant ESET polypeptide histone H3 methylase activity may be indicative of the individual having a condition associated with cellular proliferation or being predisposed i.e. having an increased susceptibility to a condition associated with cellular proliferation.

Aberrant expression may be detected at the protein level, by determining the histone H3 methylase activity of an ESET polypeptide, as described herein, for example, the presence or absence or amount of methylase activity, or at the nucleic acid level (i.e. DNA or RNA), by determining the presence of a mutant, variant or allele of an ESET gene which encodes an ESET polypeptide which has aberrant activity or which expresses aberrant i.e. abolished, reduced or increased levels of an ESET polypeptide. The presence or amount of ESET polypeptide expression may be determined by determining the presence and/or amount of mRNA encoding the polypeptide.

A histone H3 polypeptide may be a full-length histone H3 protein from a eukaryotic cell, such as a yeast or a mammal, for example a human. The term also includes fragments of the full-length protein sequence, such as fragments that comprise the lysine 9 residue, for example fragments comprising the N terminal residues of the full length protein.

An ESET polypeptide may be the full-length ESET protein of NP_{—}036564 (Version NP_{—}036564.1; GI: 6912652; PID: g6912652), or a fragment thereof which retains some or all of the methylase activity of the full-length ESET protein, for example a polypeptide which comprises some or all of the SET domain of full-length ESET. The SET domain of full-length ESET may be defined as amino acids 803 to 1272.

A SET domain may comprise three conserved motifs; NHSC, GT(x)_{S}Y and C-rich (Rhea S. et al (2000) Nature 406 593-599). An ESET polypeptide may in particular comprise one or more of these motifs.

Suitable polypeptides include any suitable variant, allele or homologue of histone H3 and/or ESET, which may be employed in such an assay. Suitable variants or derivatives of histone H3 retain the biological activity of being methylated by ESET, i.e. they include the appropriate sites of methylation by ESET, for example, lysine 9. Suitable variants or derivatives of ESET retain the histone H3 methylase activity.

A polypeptide which is an amino acid sequence variant, allele, or mutant of an amino acid sequence described herein may comprise an amino acid sequence which shares greater than about 50% sequence identity with the amino acid sequence described herein (e.g. the amino acid sequence of ESET), greater than about 60%, greater than about 70%, greater than about 80%, greater than about 90% or greater than about 95%. The sequence may share greater than about 70% similarity, greater than about 80% similarity, greater than about 90% similarity or greater than about 95% similarity with the amino acid sequence described herein (e.g. the amino acid sequence of ESET). Variations in sequence from the ESET protein o. NP_{—}036564 may for example arise through the presence of polymorphic alleles which differ between individuals within a species or through the presence of residues which are not conserved between homologous sequences from different species.

The term “ESET polypeptide” is intended to encompass such variants, derivatives, alleles and homologues of ESET (NP_{—}036564). In particular, an ESET polypeptide may comprise a SET domain having greater than 70%, greater than 75%, greater than 80%, greater than 85%, greater than 90%, greater than 95% sequence identity with the SET domain of ESET. More particularly, the ESET polypeptide may comprise a SET domain having a greater level of sequence identity with the SET domain of ESET than the SET domain of ESET has with the SET domains of known lysine 9 methylases (e.g. Suv39H1, Suv39H2 and G9A).

Amino acid similarity and identity are generally defined with reference to the algorithm GAP (Wisconsin Package, Acelerys Ltd, CA). CAP uses the Needleman and Wunsch algorithm to align two complete sequences that maximizes the number of matches and minimizes the number of gaps. Generally, the default parameters are used, with a gap creation penalty=12 and gap extension penalty=4. Use of C-AP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol. Biol. 147: 195-197), generally employing default parameters.

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Particular amino acid sequence variants may differ from a sequence described herein by insertion, addition, substitution or deletion of 1 amino acid, 2, 3, 4, 5-10, 10-20 20-30, 30-50, 50-100, 100-150, or more than 150 amino acids.

Sequence comparison may be made over the full-length of the relevant sequence shown herein, or may more preferably be over a contiguous sequence of about or greater than about 20, 25, 30, 33, 40, 50, 67, 133, 167, 200, 233, 267, 300, 333, 400 or more amino acids, compared with the relevant amino acid sequence.

Methods of obtaining agents able to modulate the histone H3 methylation activity of ESET polypeptides include methods wherein a suitable end-point is used to assess interaction in the presence and absence of a test compound.

For methylation assays, a histone H3 or ESET polypetide which is a full-length protein, truncated portion, or a portion of fused to another protein (eg. GST), or a suitable variant or derivative of any of these, may be used.

Peptide methylation assays may use peptides that comprise the region of histone H3 which is methylated.

The methylation of histone H3 may be assayed by any of a variety of procedures such as discussed below and may be adapted to high throughput screening approaches.

Fragments of a full-length protein may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art.]

As described above, peptides which include or consist of fragments of full length ESET protein are encompassed within the term ‘ESET polypeptide’ as defined herein. A fragment of a polypeptide has fewer residues than the full-length polypeptide, for example one or more, two or more, three or more, five or more or ten or more fewer residues.

Further aspects of the invention provides a fragment of the ESET protein as described herein (NP_{—}036564) having histone H3 methylase activity and a nucleic acid sequence encoding such a fragment. A suitable fragment preferably methylates histone H3 at lysine 9.

The invention also provides a vector comprising such a nucleic acid sequence, for example operably linked to a regulatory element and a host cell comprising such a vector.

The skilled person can use the techniques described herein and others well known in the art to produce large amounts of peptide or polypeptide, for instance by expression from encoding nucleic acid. Vectors comprising nucleic acid encoding an ESET polypeptide or a fragment thereof may be transformed into a suitable host cell as described above to provide for expression of the ESET polypeptide. Thus, in a is further aspect the invention provides a process for preparing a ESET polypeptide which includes cultivating a host cell transformed or transfected with an expression vector as described above under conditions to provide for expression by the vector of a coding sequence encoding the ESET polypeptide, and recovering the expressed polypeptide. Polypeptides may also be expressed in in vitro systems, such as reticulocyte lysate. The coding sequence for ESET (NP_{—}036564) is available under accession number NM_{—}012432 (Version NM_{—}012432.1; GI: 6912651).

Following production of an ESET polypeptide, it may be tested for histone H3 methytransferase activity, e.g. by determination of methylation of lysine 9 on incubation of the polypeptide with a histone H3 polypeptide.

Thus the present invention also provides a method of producing an ESET polypeptide comprising:
expression an ESET polypeptide from encoding nucleic acid; and
determining the methyltransferase activity of the expressed ESET polypeptide.

The methyltransferase activity may include the methylation of the histone H3 residue lysine 9, for example the mono-, di- or tri-methylation of lysine 9.

Performance of an assay method according to the present invention may be followed by isolation and/or manufacture and/or use of a compound, compound or molecule which tests positive for ability to modulate methylation of the appropriate residue of histone H3 and/or the methylase activity of an ESET polypeptide. Following identification of a suitable agent, it may be investigated further. Furthermore, it may be manufactured and/or used in the preparation, i.e. manufacture or formulation, of a composition such as a medicament, pharmaceutical composition or drug. These may be administered to individuals.

In various aspects, the present invention thus provides a modulator identified and/or obtained by a method of the invention, e.g. a compound which inhibits or reduces, increases or potentiates the histone H3 methylase activity of an ESET polypeptide.

Following identification of a modulator, the compound may be purified and/or investigated further and/or manufactured. A modulator may be used to obtain peptidyl or non-peptidyl mimetics, e.g. by methods well known to those skilled in the art and discussed herein. It may be used in a therapeutic context as discussed below.

One class of putative modulator compounds can be derived from the ESET polypeptide sequence and/or a ligand with which it interacts, such as histone H3. Peptide fragments of these polypeptides or alleles, mutants or derivatives of such fragments are described herein. Nucleic acid encoding such peptides, vectors and host cells containing such nucleic acid, and methods of expressing nucleic acid encoding such peptides are further aspects of the present invention.

Other agents according to the present invention useful in modulating the methylation of histone H3, and therefore one or more of its functions within the chromatin, modulate the methylase activity of the ESET polypeptide. Such agents may specifically inhibit or potentiate the ability of the ESET polypeptide to methylate the appropriate residue of histone H3. Assays and screens for such agents are provided in accordance with the present invention, along with the agents themselves and their use in modulating the methylation and thereby the function of histone H3.

An agent able to inhibit or potentiate methylation of histone H3 by an ESET polypeptide may include a compound able to affect the catalytic properties of the enzymatically active site of the methylase. An inhibitor or potentiator of methylation may interact with the ESET polypeptide within the SET methylase domain. Residues within this domain are involved in interaction with histone H3 and catalysis of the methylation. Residues outside of the domain may also be involved in interacting with histone H3 and agents which interfere with such interaction may also affect the methylation, as discussed elsewhere herein.

Agents and compounds useful in accordance with the present invention may be identified by methods which involve determining whether an agent under test increases or reduces the methylation by an ESET polypeptide of the histone H3 polypeptide.

Where the ESET polypeptide is a fragment of the full-length protein, suitable fragments are those which retain the methylase activity of the full-length polypeptide and are capable of methylating histone H3.

Where the histone H3 polypeptide is a fragment of the full-length protein, suitable fragments are those which contain lysine 9, and are themselves capable of being methylated by an ESET polypeptide. Smaller fragments, and analogues and variants of these fragment may similarly be employed, e.g. as identified using techniques such as deletion analysis or alanine scanning.

Thus, the present invention provides an ESET polypeptide, which is preferably a peptide fragment, which is able to inhibit or potentiate methylation of histone H3.

Such peptide fragments may be obtained by means of deletion analysis and/or alanine scanning of the relevant protein—making an appropriate mutation in sequence, bringing together an ESET polypeptide and a histone H3 polypeptide in the presence of the mutated fragment, and determining methylation of the histone H3 polypeptide. In preferred embodiments, the peptide is short, as discussed below, and may be a minimal portion which is able to interact with the relevant counterpart protein and inhibit or potentiate methylation.

In this context, a “fragment” of a polypeptide generally means a stretch of amino acid residues of at least about five contiguous amino acids, often at least about seven contiguous amino acids, typically at least about nine contiguous amino acids, more preferably at least about 13 contiguous amino acids, and, more preferably, at least about 20 to 30 or more contiguous amino acids. Fragments of ESET, or an ESET polypeptide, may include antigenic determinants or epitopes useful for raising antibodies to a portion of the amino acid sequence. Alanine scans are commonly used to find and refine peptide motifs within polypeptides, this involving the systematic replacement of each residue in turn with the amino acid alanine, followed by an assessment of biological activity.

Peptides in accordance with this aspect of the present invention tend to be short, and may be about 40 amino acids in length or less, preferably about 35 amino acids in length or less, more preferably about 30 amino acids in length, or less, more preferably about 25 amino acids or less, more preferably about 20 amino acids or less, more preferably about 15 amino acids or less, more preferably about 10 amino acids or less, or 9, 8, 7, 6, 5 or less in length. Peptides according to the present invention may be about 10-40 amino acids in length, about 5-10, about 10-15, about 10-20, about 10-30, about 20-30, or about 30-40 amino acids in length. Peptides which are histone H3 fragments generally include the lysine 9 residue.

The present invention also encompasses peptides which are sequence variants or derivatives of the wild-type ESET protein sequence (NP_{—}036564). Peptides which are variants of the wild-type ESET protein sequence retain the ability to modulate methylation of histone H3.

An ESET polypeptide or histone H3 polypeptide may be a peptide or polypeptide which may include an amino acid sequence which differs by one or more amino acid residues from the wild-type amino acid sequence, by one or more of addition, insertion, deletion and substitution of one or more amino acids. Thus, variants, derivatives, alleles, mutants and homologues, e.g. from other organisms (e.g. mouse or Drosophila), are included.

Preferably, the amino acid sequence shares homology (i.e. sequence similarity or identity) with a region of the ESET protein or histone H3 polypeptide as referenced herein, preferably at least about 60%, or 70%, or 75%, or 80%, or 85%, 90% or 95% homology. Thus, a peptide fragment of an ESET polypeptide or histone H3 polypeptide may include 1, 2, 3, 4, 5, greater than 5, or greater than 10 amino acid alterations such as substitutions with respect to the wild-type sequence.

A derivative of a peptide for which the specific sequence is disclosed herein may be in certain embodiments the same length or shorter than the specific peptide. In other embodiments the peptide sequence or a variant thereof may be included in a larger peptide, as discussed above, which may or may not include an additional portion of the ESET protein or histone H3 polypeptide. 1, 2, 3, 4 or 5 or more additional amino acids, adjacent to the relevant specific peptide fragment of ESET protein or histone H3 polypeptide, or heterologous thereto may be included at one end or both ends of the peptide.

Antibodies directed to the site of binding of ESET polypeptides form another class of putative modulators of ESET methylase activity. Candidate inhibitor antibodies may be characterised and their binding regions determined to provide single chain antibodies and fragments thereof which are responsible for disrupting the binding.

Antibodies may be obtained using techniques which are standard in the art. Methods of producing antibodies include immunising a mammal (e.g. mouse, rat, rabbit, horse, goat, sheep or monkey) with an ESET polypeptide. Antibodies may be obtained from immunised animals using any of a variety of techniques known in the art, and screened, preferably using binding of antibody to antigen of interest. For instance, Western blotting techniques or immunoprecipitation may be used (Armitage et al., 1992, Nature 357: 80-82). Isolation of antibodies and/or antibody-producing cells from an animal may be accompanied by a step of sacrificing the animal.

As an alternative or supplement to immunising a mammal with a peptide, an antibody specific for an ESET polypeptide may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding compound having an immunoglobulin binding domain with the required specificity.

Thus the invention covers antibody fragments, derivatives, functional equivalents and homologues of antibodies, including synthetic molecules and molecules whose shape mimics that of an antibody enabling it to bind an antigen or epitope.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP184187A, GB 2188038A or EP-A-0239400. Cloning and expression of chimeric antibodies are described in EP-A-0120694 and EP-A-0125023.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion which encodes antibody and reporter molecule. The mode of determining binding is not a feature of the present invention and those skilled in the art are able to choose a suitable mode according to their preference and general knowledge.

Antibodies may also be used in purifying and/or isolating an ESET polypeptide, for instance following production of the polypeptide by expression from encoding nucleic acid. Antibodies may be useful in a therapeutic context (which may include prophylaxis) to disrupt or enhance binding of an ESET polypeptide to histone H3 and modulate (i.e. inhibit or potentiate) the methylation, for example the mono-, di-, or tri-methylation of the lysine 9 residue. Antibodies can for instance be micro-injected into cells, e.g. at a tumour site, subject to radio- and/or chemo-therapy. Antibodies may be employed in accordance with the present invention for other therapeutic and non-therapeutic purposes which are discussed elsewhere herein.

As noted, the agent may be peptidyl, e.g. a peptide which includes a sequence as recited above, or may be a functional analogue of such a peptide.

As used herein, the expression “functional analogue” relates to peptide variants or organic compounds having the same functional activity as the peptide in question, which may interfere with the methylation of the lysine 9 residue of histone H3 by an ESET polypeptide. Examples of such analogues include chemical compounds which are modelled to resemble the three dimensional structure of the SET methylase domain in the contact area, and in particular the arrangement of the key amino acid residues.

In a further aspect, the present invention provides the use of an ESET polypeptide, in particular a peptide fragment, which is capable of methylating the lysine 9 residue of histone H3, in a method of designing a peptide or non-peptidyl mimetic, which mimetic is able to interact with the ESET polypeptide active site and modulate the methylation of the lysine 9 residue by the ESET polypeptide.

Accordingly, the present invention provides a method of designing a mimetic, for example of a histone H3 amino terminal fragment, which has the biological activity of modulating the methylation of the lysine 9 residue by an ESET polypeptide, said method comprising:
(i) analysing a compound to determine the amino acid residues essential and important for the biological activity to define a pharmacophore; and,
(ii) modelling the pharmacophore to design and/or screen candidate mimetics which modulate the methylation as described.

Suitable modelling techniques are known in the art. This includes the study of the bonding between the ESET polypeptide and histone H3 and the design of compounds which contain corresponding functional groups arranged in such a manner that they could reproduce that bonding.

The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, for instance ESET polypeptides may not be well suited as active agents for oral compositions as they tend to be quickly degraded by proteases in the alimentary canal.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of the above approach, the three-dimensional structure of a ligand and its binding partner are modelled. This can be especially useful where the ligand and/or binding partner change conformation on binding, allowing the model to take account of this the design of the mimetic.

A compound found to have the ability to affect ESET methylase activity has therapeutic and other potential in a number of contexts, as discussed. For therapeutic treatment such a compound may be used in combination with any other active compound, e.g. for anti-tumour therapy another anti-tumour compound or therapy, such as radiotherapy or chemotherapy.

An agent identified using one or more primary screens (e.g. in a cell-free system) as having ability to modulate the methylase activity of an ESET polypeptide may be assessed further using one or more secondary screens. A secondary screen may involve testing for a biological function of histone H3 methylated at the lysine 9 position such as transcriptional repression. Alternatively, a secondary screen is may involve testing for a biological function of histone H3 which is not methylated at the lysine 9 position such as transcriptional activation.

Generally, a modulator according to the present invention is provided in an isolated and/or purified form, i.e. substantially pure. This may include being in a composition where it represents at least about 90% active ingredient, more preferably at least about 95%, more preferably at least about 98%. Such a composition may, however, include inert carrier materials or other pharmaceutically and physiologically acceptable excipients. As noted below, a composition according to the present invention may include in addition to an modulator compound as disclosed, one or more other molecules of therapeutic use, such as an anti-tumour agent.

The invention further provides a method of treatment which includes administering to a patient a compound which reduces or decreases or increases or potentiates the methylation of the target residue of histone H3 by ESET polypeptide. Suitable compounds are discussed below. Exemplary purposes of such treatment are discussed elsewhere herein.

A suitable compound may, for example, reduce the methylation of the lysine 9 residue of histone H3 by an ESET polypeptide in a tumour suppressor gene or increase the methylation of the lysine 9 residue of histone H3 by an ESET polypeptide in an oncogene or tumour enhancing gene.

The invention further provides various therapeutic methods and uses of one or more compounds selected from (i) an ESET polypeptide which is able to methylate histone H3 at lysine 9; (ii) a nucleic acid encoding an ESET polypeptide which is able to methylate histone H3 at lysine 9; (iii) a modulator identified by a screening method of the present invention which is able to reduce or decrease (i.e. inhibit) or increase or potentiate (i.e. enhance) such methylation; (iv) a mimetic of any of the above compounds which can bind to histone H3 or an ESET polypeptide.

Aspects of the invention provide a compound selected from the group consisting of (i), (ii), (iii) and (iv) above for use in a method of treatment, for example a method of treating a condition associated with cellular proliferation, and the use of a compound selected from the group consisting of (i), (ii), (iii) and (iv) above in the manufacture of a medicament for use in the treatment of a condition associated with cellular proliferation.

The therapeutic/prophylactic purpose of such a method or use may be the modulation, e.g. inhibition or potentiation of the methylation of histone H3 at the residue lysine 9. As described in the introduction, methylation of lysine 9 of histone H3 may be involved in the inhibition of transcriptional activation and the promotion of chromatin silencing. This activity may have a direct effect on cellular proliferation.

Accordingly, the therapeutic/prophylactic purpose may be:
(i) Cancer treatment, which may for example be in combination with chemotherapy and/or radiotherapy;
(ii) Cancer prophylaxis;
(iii) Treatment of other proliferative disorders described herein e.g. psoriasis, cataracts, multiple myeloma.

In various further aspects, the present invention thus is provides a pharmaceutical composition, medicament, drug or other composition for such a purpose, the composition comprising one or more of compounds (i) to (iv) above, the use of such a compound in a method of medical treatment, a method comprising administration of such a compound to a patient, e.g. for treatment (which may include preventative treatment) of a medical condition, e.g. a condition associated with a defect or disorder in transcriptional control, DNA replication, or cell cycle control, e.g. for treatment of a disorder or condition associated with cellular proliferation such as restenosis, psoriasis, cataracts, multiple myeloma and cancer, preferably all types of solid cancers and malignant lymphomas and especially leukaemia, skin cancer, bladder cancer, breast cancer, uterus cancer, ovary cancer, prostate cancer, lung cancer, colon cancer, pancreas cancer, renal cancer, stomach cancer and cerebral cancer, use of one or more of compounds (i) to (iv) above in the manufacture of a composition, medicament or drug for administration for such a purpose, e.g. for treatment of a condition associated with proliferation, and a method of making a pharmaceutical composition comprising admixing such a compound with a pharmaceutically acceptable excipient, vehicle or carrier, and optionally other ingredients.

The compounds may be used as sole active agents or in combination with one another or with any other active compound, e.g. for anti-tumour therapy another anti-tumour compound or therapy, such as radiotherapy or chemotherapy.

Whatever the compound used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A compound or composition may be administered alone or in combination with other treatments, either simultaneously or sequentially dependent upon the condition to be treated, e.g. cancer.

Pharmaceutical compositions according to the present invent-ion, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as requited.

Liposomes, particularly cationic liposomes, may be used in carrier formulations.

Examples of techniques and protocols mentioned above can be found in Remington's Pharmaceutical Sciences, 16th edition, Osol, A. (ed), 1980.

The compound or composition may be administered in a localised manner to a tumour site or other desired site or may be delivered in a manner in which it targets tumour or other cells.

Targeting therapies may be used to deliver the active compound more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering such compounds directly, they may be produced in the target cells by expression from an encoding nucleic acid introduced into the cells, e.g. from a viral vector. The vector may be targeted to the specific cells to be treated, or it may contain regulatory elements-which are switched on more or less selectively by the target cells.

Nucleic acid encoding the compound e.g. a peptide or polypeptide able to modulate, e.g. inhibit or potentiate, the methylation of the lysine 9 residue of histone H3, may thus be used in methods of gene therapy, for instance in treatment of individuals, e.g. with the aim of preventing or curing (wholly or partially) a disorder.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

A peptide or other compound having an ability to modulate the methylation of the lysine 9 residue of histone H3 by an ESET polypeptide, or a nucleic acid molecule which encodes a peptide having that ability, may be provided in a kit, e.g. sealed in a suitable container which protects its contents from the external environment. Such a kit may include instructions for use.

In still further aspects the present invention provides for the purification of an ESET polypeptide, or for the purification of a histone H3 polypeptide. The invention also provides for a purified ESET polypeptide. The purified polypeptide may be about 10% pure, more preferably about 20% pure, more preferably about 30% pure, more preferably about 40% pure, more preferably about 50% pure, more preferably about 60% pure, more preferably about 70% pure, more preferably about 80% pure, more preferably about 90% pure, more preferably about 95% pure, or substantially pure.

In another aspect the present invention provides a method of purifying an ESET polypeptide, the method including contacting the ESET polypeptide with histone H3 polypeptide.

A mixture of material including an ESET polypeptide may be contacted against immobilised histone H3 polypeptide (e.g. immobilised either covalently or non-covalently such as via a specific binding molecule such as streptavidin or biotin) and molecules which do not bind to the histone H3 polypeptide are washed off.

Following purification, the ESET polypeptide may be used as desired, e.g. in an assay for an agent which modulates its activity, e.g. binding, in raising or obtaining a specific antibody or other binding molecule, or in a therapeutic context.

Various further aspects and embodiments of the present invention will be apparent to those skilled in the art in view of the present disclosure.

Certain aspects and embodiments of the invention will now be illustrated by way of example and with reference to the figure described below.

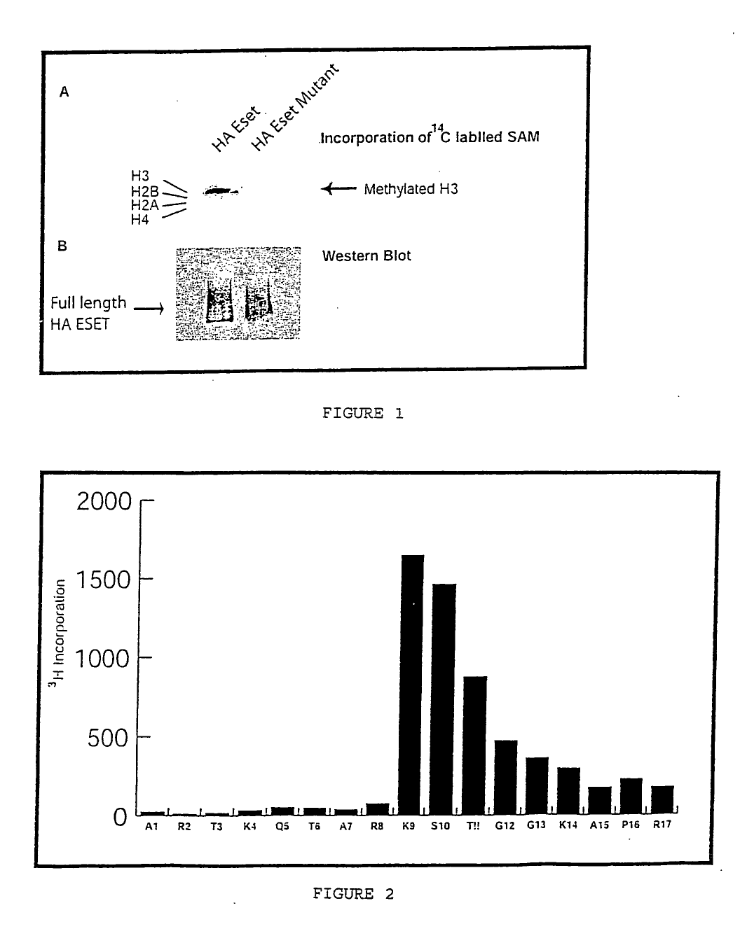

FIG. 1 shows methyltransferase activity of ESET is absent from a point mutant (C1226A) mutated in the SET domain of ESET.

A: Full-length human HA tagged ESET and an HA tagged ESET point mutant (C1226A) were immunoprecipitated from transfected 293T cells using anti-HA antibody. Immunopreciptates were then used in standard histone methyltransferase assay using ¹⁴C radiolablled SAM and reaction buffer (50M Tris pH 8.5, 75 mM NaCl, 0.5% NP40, 1 mM DTT, 5 mM MgCl). ESET specifically is methylates histone H3 and the point mutant is inactive.

B: HA western blots of 293T extracts showing expression of both mutant and wild type ESET.

FIG. 2 shows that ESET specifically methylates lysine 9 of histone H3.

Methylation reactions using immunoprecipitated ESET were carried out as in FIG. 1, using ³H labelled SAM. H3 histone was then subjected to N-terminal Edman degradation and each amino acid fraction collected. Amino acid fractions were subjected to scintillation analysis, the results of which are shown in the Figure, showing that ESET specifically methylates lysine 9.

### EXPERIMENTAL

Materials and Methods

Immunoprecipitation of Human ESET Protein

Human ESET was tagged at the N-terminus with a HA epitope from the influenza virus. The protein was expressed in human 293T cells following calcium phosphate transfection of the ESET expression plasmid. The tagged protein with the expected size was detected in total extract by western blot with anti-HA antibody (MAP, mouse, Roche 12CA5) 1:1000 dilution. Cells expressing HA-ESET were lysed in IPH buffer (50 mM Tris.Cl pH8.0, 150 mM NaCl, 5 mM EDTA, 0.5% (v/v) NP-40) and 3 μg of the anti-HA antibody was added. Fifteen μl of a 50% slurry of protein A bound to agarose was added and the reaction was slowly rotated overnight at 4° C. The following day the immuno complex was centrifuged to pellet the complex. The pellet was then washed 4 times with 1 ml of IPH buffer.

Methyltransferase Assay

The enzymatic assays were performed on a mixture of soluble histones from calf thymus (H2A, H2B, H3 and H4), Sigma; or recombinant xenopus H3. For each reaction, 60 μg of calf histones or 20 μg of recombinant H3 were used. The following mixtures were prepared:
1) 10 μl of immunoprecipitated ESET on beads+30 μl of methylation buffer+2 μl of ¹⁴C-SAM (NEC-363 adenosyl-L-methionine, S-(methyl-¹⁴C).
2) 10 μl of immunoprecipitated ESET on beads+30 μl of methylation buffer+3 μl of soluble histones (10 μg)+2 μl of ¹⁴C-SAM (NEC-363 adenosyl-L-methionine, S-(methyl-¹⁴C).
3) 10 μl of immunoprecipitated ESET on beads+30 μl of methylation buffer+4 μl of recombinant xH3 (10 μg)+2 μl of ¹⁴C-SAM (NEC-363 adenosyl-L-methionine, S-(methyl-¹⁴C).
4) 10 μl of IgG sepharose beads (previously equilibrated in methylation buffer)+30 μl of methylation buffer+3 μl of soluble histones (10 μg)+2 μl of ¹⁴C-SAM (NEC-363 adenosyl-L-methionine, S-(methyl-¹⁴C)
5) 10 μl of IgG sepharose beads (previously equilibrated in methylation buffer)+30 μl of methylation buffer+4 μl of recombinant xH3 (20 μg)+2 μl of ¹⁴C-SAM (NEC-363 adenosyl-L-methionine, S-(methyl-14C).

Methylation Buffer: 50 mM Tris-HCl pH 8.5; 75 mM NaCl, 5 mM MgCl₂, 1 mM DTT, 0.5% NP-40.

The microfuge tubes were incubated at 30° C. during 2 hours with shaking. After that, 15 μl of 4× (protein sample buffer) were added to each reaction (the total liquid vol. of each reaction was about 45 μl). The samples were boiled for 3 minutes. The histones were separated in a 1.5 mm, 20% acrylamide gel, run at 200 V during 1 hour and 15 minutes and transferred to nitro-cellulose membranes by standard procedures. The nitro-cellulose membrane was exposed to Kodak Biomax MS film with Biomax Transcreen-LE intensifying screen at −80 C during 36-48 h.

Mapping of Site of Methylation within Histone H3

Histone H3 was methylated by immunopreciptated ESET as described herein with a ¹⁴C-methyl group. The methylated residue was then identified using N terminal sequence analysis as described in Rein D. and Speicher D. Current Protocols in Protein Science (1997) 11.10.01-11.10.38.

Briefly, a stained protein band was excised from a PVDF membrane, sonicated in a both sonicator and sequenced using a Hewlett-Packard Model G 100SA sequencing system. Sequencing was carried out according to the manufacturer's instructions and PTH (phenylthiohydantoin) amino acids from each sequencer cycle were separated by in-line HPLC to provide sequence data.

Results

Human ESET is a Histone Methyltransferase (HMT)

HA tagged ESET was purified from human 293T cells by anti-HA antibody immunoprecipitation. Western blotting revealed a is strong band in the immunoprecipitate corresponding to full-length HA-ESET.

The immunoprecipitated HA-ESET was assayed for methyltransferase activity on 10:g of a mixture of calf thymus soluble histones (H2A, H2B, H3 and H4) or 10:g of recombinant xenopus histone H3 in the presence of (¹⁴C-Me) S-adenosyl methionine. Autoradiograms were developed after 48 hours exposure.

No methylation was observed for HA-ESET in the absence of calf or recombinant histones.

HA-ESET was observed to methylate recombinant Xenopus histone H3. When the preparation of histones H2A, H2B, H3 and H4 was used, HA-ESET was observed to specifically methylate histone H3.

The Methyltransferase Activity of ESET is Specific for Lysine 9 of Histone H3

For each assay, 10 μg of recombinant Xenopus H3 histone and immunoprecipitated HA-ESET were incubated in the presence of (¹⁴C-Me) S-adenosyl methionine. Autoradiograms showing the presence of ¹⁴C label were developed after 48 hours. The presence of substrates on the gel was confirmed by Ponceau staining.

No methylation was observed in the absence of a histone H3 substrate.

HA-ESET was observed to methylate full length Xenopus histone H3.

Mapping of the Methylation Site within the H3 Amino Tail

Xenopus H3 histone, labelled with a ¹⁴C-methyl group by immunoprecipitated ESET as described above, was sequenced by sequential Edman degradation and fractions corresponding to each amino acid cycle were collected and counted by scintillation counting. The results are shown in FIG. 2. The N terminal sequence of Xenopus histone H3 is shown underneath corresponding fraction.

These results show that the lysine 9 residue of histone H3 is specifically methylated by ESET. No methylation of lysine 4 is observed.

Methylation of histone H3 by ESET may therefore play an important role in the regulation of transcriptional activity and the control of cellular proliferation. Manipulation of the ESET histone methyltransferase activity may therefore be used in the treatment of conditions associated with cellular proliferation, such as cancer.

1. A method for obtaining a compound which modulates the interaction of histone H3 with a ESET polypeptide, the method including:
(a) contacting a ESET polypeptide and a histone H3 polypeptide in the presence of a test compound; and,
(b) determining interaction between the histone H3 polypeptide and the ESET polypeptide.

2. A method for obtaining a compound which modulates the histone H3 methylase activity of a ESET polypeptide, the method including:
(a) contacting a ESET polypeptide and a test compound; and,
(b) determining the histone H3 methylase activity of the ESET polypeptide.

3. A method according to claim 2 wherein histone H3 methylase activity is determined by detecting the methylation of lysine 9 of a histone H3 polypeptide.

4. A method according to claim 1 or 2 wherein the methylation is detected by contacting the histone H3 polypeptide with a specific binding member which binds specifically to histone H3 methylated at Lys 9.

5. A method according to claim 1 or 2 wherein the methylation is detected by contacting the histone H3 polypeptide with a specific binding member which binds specifically to histone H3 unmethylated at Lys 9.

6. A method according to claim 1 or 2 further comprising; testing for a biological function of histone H3 methylated at Lys 9.

7. A method according to claim 6 wherein said biological function is transcriptional repression.

8. A method according to claim 1 or 2 further comprising; testing for a biological function of histone H3 unmethylated at Lys 9.

9. A method according to claim 8 wherein said biological function is transcriptional activation.

10. A method according to claim 1 or 2 comprising identifying a test compound which modulates the histone H3 methylase activity of an ESET polypeptide.

11. A method according to claim 10 comprising isolating, purifying and/or manufacturing said test compound.

12. A method according to claim 11 comprising the step of modifying the compound to optimise the pharmaceutical properties thereof.

13. A method according to claim 11 comprising formulating said test compound in a pharmaceutical composition with a pharmaceutically acceptable excipient, vehicle or carrier.

14. A compound which modulates ESET activity and is obtained by a method of claim 1 or 2.

15. A method of producing a pharmaceutical composition comprising;
identifying a compound which modulates the activity of an ESET polypeptide using a method according to claim 1 or 2; and,
admixing the compound identified thereby with a pharmaceutically acceptable carrier.

16. A method according to claim 15 comprising the step of modifying the compound to optimise the pharmaceutical properties thereof.

17. A method for preparing a pharmaceutical composition for treating a condition associated with cellular proliferation comprising;
i) identifying an agonist/antagonist of a ESET polypeptide
ii) synthesising the identified compound, and;
iii) incorporating the compound into a pharmaceutical composition.

18. A method of producing an ESET polypeptide comprising:
(a) causing expression from nucleic acid which encodes an ESET polypeptide in a suitable expression system to produce the polypeptide recombinantly;
(b) testing the recombinantly produced polypeptide for ability to methylate lysine 9 of histone H3.

19. A ESET polypeptide fragment having histone H3 methylase activity.

20. An ESET polypeptide having histone H3 methylase activity for use in a method of treatment of the human or animal body.

21. An isolated nucleic acid encoding an ESET polypeptide having histone H3 methylase activity for use in a method of treatment of the human or animal body.

22. Use of a compound according to claim 14, an ESET polypeptide according to claim 20 or a nucleic acid according to claim 21 in the manufacture of a medicament for use in the treatment of a condition associated with cellular proliferation.

23. A method of treatment of a condition associated with cellular proliferation comprising administering to an individual one or more agents selected from the group consisting of a compound according to claim 14, an ESET polypeptide having histone H3 methylase activity and a nucleic acid encoding an ESET polypeptide having histone H3 methylase activity.
